(19) United States
(12) Patent Application Publication
(10) Pub. No.: US 2006/0155161 A1
(43) Pub. Date: Jul. 13, 2006

(54) Artificial myocardial device assisting motion of heart

(76) Inventors: Tomoyuki Yambe, Miyagi (JP); Yasuyuki Shiraishi, Miyagi (JP)

Correspondence Address: Ralph A. Dowell of DOWELL & DOWELL P.C., 2111 Eisenhower Ave, Suite 406, Alexandria, VA, 22314, US

(21) Appl. No.: 11/311,430

(22) Filed: Dec. 20, 2005

Related U.S. Application Data
(63) Continuation of application No. PCT/JP04/01695, filed on Feb. 17, 2004.

(30) Foreign Application Priority Data
Jun. 20, 2003 (JP) 2003-176588

(51) Int. Cl. A61M 1/12

(52) U.S. Cl. 600/17; 623/3.23; 623/3.28

(57) ABSTRACT

A container has a diaphragm in abutment with an outer wall of a heart, and contains silicone oil. An actuator driven by a motor applies pressure on the silicone oil in the container to drive the diaphragm. A controller controls the operation of the motor.

### RELATED APPLICATIONS

This application is a continuation of PCT application no. PCT/JP2004/001695, filed Feb. 17, 2004.

### BACKGROUND

1. Field

The present invention relates to an artificial myocardial device attached to, for example, a heart and assisting motion of heart.

2. Description of the Related Art

Artificial hearts that replace for a defective heart or assist functions of a heart have been developed. A whole artificial heart is a system to be implanted in place of a heart that is ablated, and an auxiliary artificial heart is a pump system compensating part of motion of heart.

In Europe and the United States, clinical application of whole artificial heart has already started. However, a whole artificial heart has generally a large device profile so that it is difficult to be applied to Japanese. Auxiliary artificial hearts are classified into extracorporeal devices and implant devices. Although extracorporeal devices are already used in Japan, a patient with an auxiliary artificial heart is hard to move away from a driver of the auxiliary artificial heart and hence forced to be confined to bed. Auxiliary artificial hearts of implant type have been developed in Europe and the United states. These are also too large in profile to be applied to Japanese who are generally small in stature.

In view of the above, also developed is an auxiliary artificial heart that utilizes a centrifugal pump to realize miniaturization (Wieselthaler G M, Schima H, Hiesmayr M, Pacher R, Laufer G, Noon G P, DeBakey M, Wolner E. First clinical experience with the DeBakey VAD continuous-axial-flow pump for bridge to transplantation. Circulation. 2000 Feb. 1; 101(4): 356-9). This device, however, is unphysiologic because it is difficult to generate pulses and the blood circulation lacks pulses. Also these artificial hearts and auxiliary artificial hearts have risks of thrombus formation due to contacting with blood, and hence the patient encounters the risk of cerebral stroke.

As described above, the conventional whole artificial hearts and auxiliary artificial hearts encounter the problems of thrombus formation. Briefly, in conventional devices, blood is introduced into a pump via a pipe from a blood vessel or the like, and then flowed back to the blood vessel from the pump via the pipe. Blood necessarily coagulates on contacting an artificial material such as a pipe or a pump. In order to prevent coagulation of blood, various antithrombogenic materials are developed and measures for preventing thrombus formation by keeping the blood flow are taken. However, perfect prevention is still difficult.

In order to prevent thrombus, it is necessary for an artificial heart to keep the blood flow while constantly beating. However, there is a great problem in improving the durability of artificial heart. For example, a human heart beats about 100,000 times per day. Accordingly, development of a miniaturized artificial heart which is durable to 100,000 beats per day is requested.

### SUMMARY

In an embodiment of the present invention, an artificial myocardial device comprises: a container having a diaphragm in abutment with an outer wall of a heart and accommodating therein a fluid; a pressure generator connected with the container, the pressure generator applying a pressure on the fluid in the container to drive the diaphragm; a motor which drives the pressure generator; and a controller which controls the motor.

In the artificial myocardial device, the pressure generator may include: a cylinder, a piston which is movable in the cylinder in a reciprocating manner; and a ball screw which converts rotational motion of the motor into reciprocating motion to operate the piston.

The artificial myocardial device may further comprise: a first sensor provided near the diaphragm, the first sensor detecting a pressure from the heart; and a second sensor which detects a flow rate of the blood, wherein the controller controls operation of the motor in accordance with detection output signals from the first and second sensors.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a structural view showing an artificial myocardial device attached in a body;

FIG. 2 is a structural view showing one example of an artificial myocardial device;

FIG. 3 is a structural view showing one example of a control system of an artificial myocardial device;

FIG. 4 is a flowchart showing one example of operation of a controller.

### DETAILED DESCRIPTION

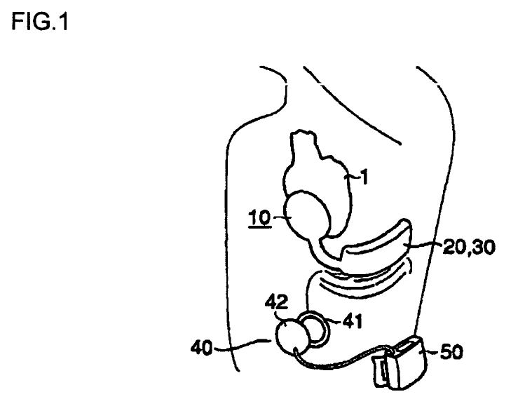

In the following, preferred embodiments of the present invention will be described with reference to the drawings. FIG. 1 shows one example of an artificial myocardial device according to one embodiment of the present invention. The artificial myocardial device is a system of e.g., implant type that directly pushes a heart 1 from outside by fluid pressure, and is an artificial myocardial device composed of a so-called hydraulic actuator. This artificial myocardial device includes: an artificial myocardium 10 attached to outside of a cardiac chamber; an actuator 20 which hydraulically actuates the artificial myocardium 10; a controller 30 which controls operation of the actuator 20; a transdermal energy transmitting system 40 which supplies the controller 30 with energy from outside of the body; and a power unit 50. The artificial myocardium 10, the actuator 20, the controller 30, and a receiver 41 of the transdermal energy transmitting system 40 are provided inside the body, and the power unit 50 and the transmitter 42 of the transdermal energy transmitting system 40 are provided outside the body. The artificial myocardium 10 is for example, sewed on the outside of the heart, and the actuator 20 and the controller 30 are located in an intercostal space.

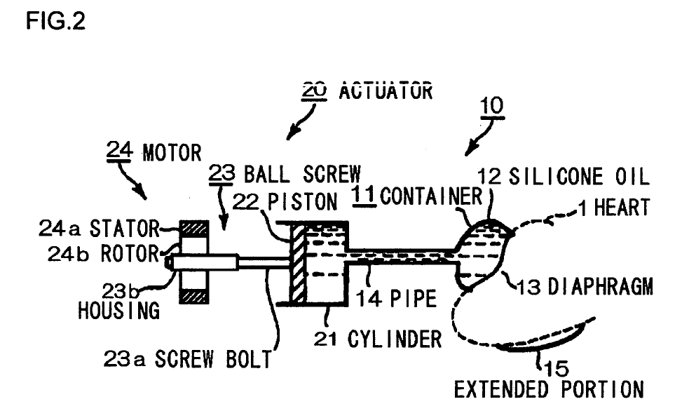

As shown in FIG. 2, the artificial myocardium 10 includes: a container 11 filled with a fluid such as silicone oil 12; a diaphragm 13 provided in the container 11, having a shape which is variable with the pressure of fluid; a cylinder 21 constituting the actuator 20 connected via a pipe 14; a piston 22 provided within the cylinder 21; a ball screw 23 which drives the piston 22; and a motor 24 which drives the ball screw 23.

The container 11, the pipe 14, the cylinder 21, and the piston 22 are made of materials such as polycarbonate that have desired rigidity and are never rejected by organisms. The diaphragm 13 is made of a material, e.g., silicon rubber that is flexible and little changes with age, and never rejected by organisms. In the state that the container 11 is attached to a cardiac chamber, the diaphragm 13 is brought into close contact with an outer wall of the cardiac chamber, and pushes the cardiac chamber by pressure of fluid. Furthermore, the container 11 partly has an extended portion 15 extended to a reverse side of the cardiac chamber. The extended portion 15 and the diaphragm 13 securely hold the heart 1. The motor 24, the ball screw 23, the cylinder 21, and the piston 22 are placed in an intercostal space, for example, with the controller 30.

The ball screw 23 converts rotational motion of the motor 24 into reciprocating motion by means of a ball screw. As is well-known, the ball screw 23 has a housing 23b of for example, a cylindrical shape which accommodates a screw bolt 23a and a ball (not shown). The ball in the housing 23b is fitted with the screw bolt 23a. The motor 24 has a stator 24a and a rotor 24b provided within the stator 24a. The rotor 24a is connected to the housing 23b, and as the rotor 24a rotates, the housing 23b rotates concurrently and the screw bolt 23a linearly moves. The screw bolt 23a reciprocatingly moves in accordance with the rotation direction of the rotor 24a. When the ball screw 23 is driven by the motor 24 in the manner as described above, the piston 22 reciprocatingly moves and hydraulic pressure is generated in the cylinder 21. The hydraulic pressure generated within the cylinder 21 is transmitted to the container 11 via the pipe 14. This hydraulic pressure drives the diaphragm 13, and the cardiac chamber is pushed.

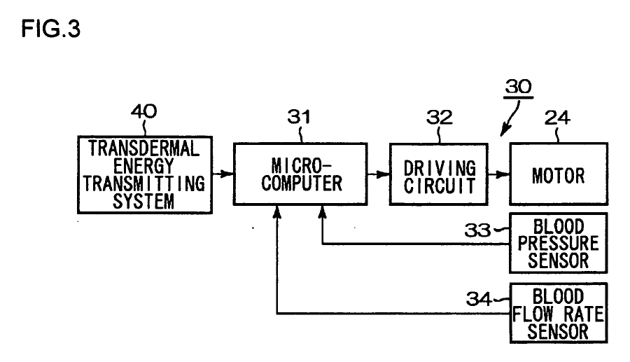

FIG. 3 shows one example of the controller 30. The controller 30 includes a microprocessor 31, for example. To the microprocessor 31, a driving circuit 32 for driving the motor 23, for example, a blood pressure sensor 33, and a blood flow rate sensor 34 are connected. The blood pressure sensor 33 is implemented by, for example, a pressure sensor. The blood pressure sensor 33 disposed, for example, between the diaphragm 13 and the cardiac chamber detects a pressure from the heart. The blood flow rate sensor 34 is implemented by, for example, an ultrasonic blood flow sensor. The blood flow rate sensor 34 disposed, for example, on an outer wall of a blood vessel near the heart detects a blood flow rate.

The transdermal energy transmitting system 40 is composed of the transmitter 42 connected to the power unit 50 and the receiver 41 connected to the controller 30. The transdermal energy transmitting system 40 has a well-known arrangement, and the transmitter 42 and the receiver 41 each include a coil. These coils are electromagnetically coupled with each other through skin. Electric energy outputted from the power unit 50 is transmitted after converted into an electromagnetic signal by the coil of the transmitter 42, and received by the coil of the receiver 41. The receiver 41 converts the received electromagnetic signal into electric energy and supplies the controller 30 with the electric energy.

The microprocessor 31 controls the driving circuit 32 in accordance with the signals supplied from the blood pressure sensor 33 and blood flow rate sensor 34 to control the operation of the motor 24.

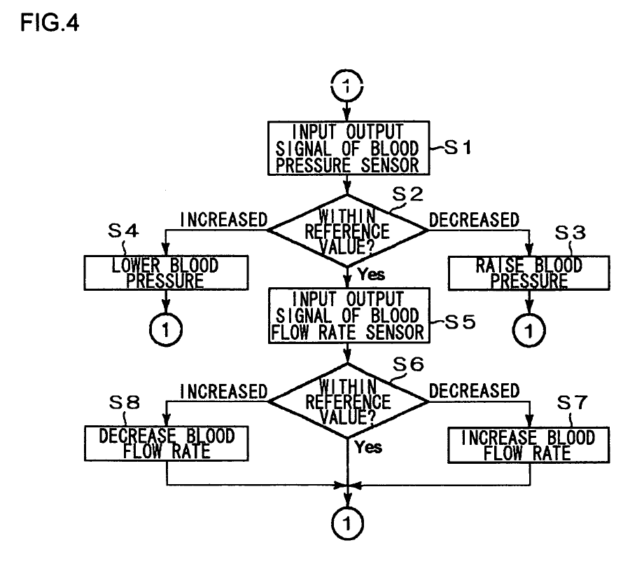

FIG. 4 shows one example of operation of the microprocessor 31. The microprocessor 31 compares output signals from the blood pressure sensor 33 and the blood flow rate sensor 34 with a reference value. If it is determined that the blood pressure decreases below the reference value, for example, based on an output signal of the blood pressure sensor 33 (S1, S2), the microprocessor 31 operates the driving circuit 32 to drive the motor 24 to increase the width of the reciprocating motion of the ball screw 23 for raising the blood pressure (S3). On the other hand, if it is determined that the blood pressure increases above the reference value, for example, based on an output signal of the blood pressure sensor 33, the microprocessor 31 operates the driving circuit 32 to drive the motor 24 to decrease the width of the reciprocating motion of the ball screw 23 for lowering the blood pressure (S4).

When the output signal of the blood flow rate sensor 34 is less than the reference value (S5, S6), the microprocessor 31 controls the operation of the motor 24 by means of the driving circuit 32 to increase the blood flow rate by shortening the period of the reciprocating motion of the ball screw 23 (S7). On the other hand, when the output signal of the blood flow rate sensor 34 is higher than the reference value, the operation of the motor 24 is controlled by the driving circuit 32 to decrease the blood flow rate by elongating the period of the reciprocating motion of the ball screw 23 (S8).

The artificial myocardial device need not always operate, but operates as needed in accordance with the change in blood pressure and blood flow rate.

According to the artificial myocardial device of the above embodiment, by generating hydraulic pressure by means of the motor 24 and the actuator 20, and driving the diaphragm 13 provided in the container 11 by the hydraulic pressure, the myocardium is pushed from outside and motion of the heart is assisted. Therefore, every part constituting the artificial myocardial device does not contact the blood circulating through the heart. Therefore, it is possible to prevent formation of thrombus.

Additionally, the artificial myocardial device is not of a pump shape that directly circulates the blood, and the artificial myocardium 10 composed of the container 11 and the diaphragm 13 has such a dimension that can be attached to a part of an outer wall of a cardiac chamber. Also the actuator 20 and the controller 30 for driving the diaphragm 13 may be miniaturized to such an extent that they can be placed in an intercostal space. Therefore, the artificial myocardial device can be readily placed in a body regardless of the size of the body.

The actuator 20 has the cylinder 21 filled with the silicone oil 12, the piston 22, the ball screw 23 and the motor 24, drives the piston 22 by means of the motor 24 and the ball screw 23 to generate hydraulic pressure, and drives the diaphragm 13 provided in the container 11 by means of the hydraulic pressure, thereby pushing the myocardium. Therefore, by controlling the direction, speed and torque of rotation of the motor 24, it is possible to set desired required pulse, blood pressure and blood flow rate.

Since the motor 24 and the ball screw 23 exhibit high power factor and efficiency, it is possible to readily and accurately control the mechanical assist for the heart with reduced energy. The motor 24 and the ball screw 23 in their non-driven states are free from the positional change of the piston 22. Therefore, they move freely in relation to loads by natural heartbeats, so that it is possible to reduce the burden on the heart.

Additionally, the artificial myocardial device need not always operate and maintain the blood flow in order to prevent formation of thrombosis as is conventional artificial hearts, but operates as necessary. Therefore, it is possible to improve the durability of driving parts and increase the durable years of the artificial myocardial device.

For the energy for driving the artificial myocardial device, the wireless transdermal energy transmitting system 40 is used. Accordingly, wiring that penetrates the skin from outside of the body is not required, which leads an advantage of no risk of bacterium infection.

While the preferred embodiments of the present invention have been described using specific terms, such description is for illustrative purposes only, and it is to be understood that changes and variations may be made without departing from the scope of the appended claims.

What is claimed is:

1. An artificial myocardial device comprising:
a container having a diaphragm in abutment with a part of an outer wall of a cardiac chamber of a heart and an extended portion extending to a reverse side of the cardiac chamber, the container accommodating therein a fluid;
a pressure generator connected to the container, the pressure generator driving the diaphragm by applying pressure on the fluid in the container;
a motor which drives the pressure generator; and
a controller which controls operation of the motor in accordance with an output signal from a predetermined sensor.

2. The artificial myocardial device according to claim 1, wherein
the pressure generator comprises:
a cylinder;
a piston which is movable in the cylinder in a reciprocating manner; and
a ball screw which converts rotational motion of the motor into reciprocating motion to operate the piston.

3. The artificial myocardial device according to claim 1, further comprising:
a first sensor disposed near the diaphragm, the first sensor detecting a pressure from the heart; and
a second sensor which detects flow rate of the blood,
wherein the controller operates the motor when at least either of the pressure and the flow rate exceeds a predetermined reference value in accordance with detection output signals of the first and second sensors.
